(19) United States
(12) Patent Application Publication
(10) Pub. No.: US 2025/0145862 A1
(43) Pub. Date: May 8, 2025

(54) METHOD TO INCREASE THE RATE OF VISCOSITY DEGRADATION OF A GELATIN, AND GELATIN COMPOSITION

(71) Applicant: GELITA AG, Eberbach (DE)

(72) Inventors: Eric YEZDIMER, Sioux City, IA (US); Michelle A. MONTGOMERY, Jefferson, SD (US); Ulrich MACH, Ulm (DE)

(21) Appl. No.: 19/013,970

(22) Filed: Jan. 8, 2025

Related U.S. Application Data
(63) Continuation of application No. PCT/EP2023/070477, filed on Jul. 24, 2023.
(60) Provisional application No. 63/391,880, filed on Jul. 25, 2022.

(51) Int. Cl. C09H 5/00

(52) U.S. Cl. CPC C09H 5/00

(57) ABSTRACT

The invention relates to a method to increase the rate of viscosity degradation of a gelatin, comprising: providing an aqueous solution of gelatin; combining the aqueous solution of gelatin with about 2 to about 20% reactive peptides, wherein the reactive peptides comprise collagen peptides with an average molecular weight of about 100 to about 5,000 Da and/or amino acids; adjusting a pH of the aqueous solution about 3.4 to about 8.0; heating the aqueous solution to a temperature from about 60 to about 150° C.; and holding the aqueous solution at said temperature for a period of time sufficient to degrade the viscosity of the solution to be in a range from about 15 to about 25 mP, when measured at a total dissolved protein concentration of 6.67% w/w, at a temperature of 60° C. and at a pH of 5.3 to 5.5.

### CROSS-REFERENCE TO RELATED APPLICATIONS

This patent application is a continuation of International Patent Application No. PCT/EP2023/070477, filed Jul. 24, 2023, which claims priority to U.S. Provisional Patent Application No. 63/391,880, filed Jul. 25, 2022, each of which is hereby incorporated by reference in its entirety.

### BACKGROUND OF THE INVENTION

Gelatin is an important component in the manufacturing of both hard capsules, soft capsules and tablet coatings for the pharmaceutical and nutraceutical markets. Commercially manufactured gelatin is comprised of a mixture of denatured and partially hydrolyzed collagen molecules. Many collagen types are known to exist, with Type I collagen being the major source of commercially available gelatin and is prevalently found in connective tissues like tendons, skin and bone. The protein chains of Type I collagen are comprised of approximately 1014 amino acids and have a molecular weight of approximately 100 kDa. These molecules are referred to as a chains. α chains are comprised of a large fraction of glycine, proline and hydroxyproline with these three amino acids accounting for roughly 50 to 60% of the mass of the protein. Three a chain proteins form linear helixes that are approximately 300 nm long and several of these elements are bundled together to form collagen fibrils. The fibrils are chemically bound together through native crosslinks. These native crosslinks are produced in vivo within the animal by an enzymatic reaction that converts bioselected lysine or hydroxylysine side chains into allysine or hydroxyallysine. The aldehydes of these enzymatically modified amino acids are then free to nonenzymatically react with the primary amines of other lysine or hydroxylysine residues located at selective positions along neighboring collagen proteins.

The commercial extraction and purification of gelatin from animal tissues and bones typically involve multiple process steps including raw material pretreatment, aqueous extraction, filtration, purification, concentration, drying, milling and blending. The details of these process steps have been taught elsewhere (see Ref A), Ref B)). Acidic pretreatments produce Type A gelatins which have isoelectric points of around 7 to 9. Alkaline pretreatments produce Type B gelatins where the glutamine and asparagine amino acids are converted into glutamic and aspartic acid. Type B gelatins can have isoelectric points of around 4.7 to 5.4. Interim isoelectric points between those of Type A and Type B gelatins can also be obtained depending on the strength of the alkaline pretreatment.

Commercial gelatin sold for use in the pharmaceutical and nutraceutical markets is normally provided as a dried powder or granular with a moisture content of between 9 to 12%. Actual gelatin compositions sold as individual lots are usually prepared as a blend of several individual dried batches. These dried batches can be produced from a set of specific gelatin extracts or from material collected from a continuous gelatin extraction process taken during a set time interval. The blend formulation is determined by measuring various physical and chemical properties of each batch and then combining those batches in selected ratios to produce a final lot that meets a required set of properties for a specific application or customer specification. The properties typically considered in lot preparation include gel strength (e.g. Bloom), viscosity, viscosity breakdown, pH, optical transmittance, ash and specific inorganic cation and anion concentrations. The viscosity of a gelatin solution generally correlates with the average molecular weight (FIG. 1) to a first order approximation. Significant variations in the average molecular weight, however, can yield similar solution viscosities as this physical property also depends on other chemical properties such as pH, isoelectric point, salt content, etc. Gel strength also correlates with higher average molecular weight, although the contribution from a chain sized molecules usually dominants as linear gelatin molecules of this size can form stronger helical structures in the gelled state.

Within the gelatin manufacturing industry there is a need to balance the physical qualities and quantities of gelatin produced from a given raw material with the qualities and quantities required from the market. Gelatin is usually extracted from collagen source material in a series of extracts performed with sequentially increasing temperatures. The characteristic physical properties of the processed gelatin thus changes with each successive extract. To maximize the quantities of sellable gelatin manufactured it is often advantageous to degrade the gel strength and/or viscosity of a particular gelatin extract to better align production output to the available sales volumes in the marketplace.

Gelatin undergoes both acid and alkaline catalyzed hydrolysis, with the more extremes of pH producing faster hydrolyzation reactions. Higher temperatures increase the rate of hydrolysis. The pH of extraction affects the ratio of gel strength to viscosity and this ratio can significantly influence the suitability of a gelatin for a particular application. pH also can greatly influence the extractability of gelatin from a raw material. Likewise, in many practical situations the reduction of gel strength and/or viscosity is limited to a thermal treatment. Thermal degradation however can be quite time consuming, often requiring many hours to reach the desired physical properties. In addition, the heating of large volumes of dilute aqueous gelatin solutions requires a significant amount of energy and increases production costs. Introducing additional hold times into a gelatin manufacturing process is also undesirable as it can limit plant capacities and require the installation of large, costly storage vessels.

In addition, a key factor determining the performance of different gelatin compositions in hard or soft capsule applications is the amount of high molecular weight gelatin molecules present in gelatin lot. Large gelatin molecules are referred to in the industry as microgel and are generally defined as crosslinked gelatin chains with a molecular weight of ≥400 kDa (e.g., four or more a chains crosslinked together). The amount of microgel present in a gelatin composition generally correlates with solution viscosity to a first order approximation (FIG. 2), however within a specified viscosity range significant variations in the amount of microgel can occur. For example, for commercial lots of limed bovine bone gelatin with a 6.67%/60° C. viscosity of 40 mP, the amount of microgel present can vary by roughly a factor of two and can have a significant effect on the performance of those gelatin compositions in hard or soft capsule applications.

It is known in the capsule industry that higher percentages of gelatin microgel are associated with a higher occurrence of capsule dissolution failures. Capsule dissolution failures stem from additional chemical crosslinking reactions that can occur between gelatin molecules after capsule manufacture Ref) F. These reactions are often dependent on the chemistry of the capsule fill ingredients, although the direct crosslinking between gelatin molecules is also possible under low moisture conditions. When chemical crosslinking occurs within the shell of a gelatin capsule the molecular weight of the gelatin molecules is increased. As the molecular weight and covalent bonded connectivity is increased within the capsule shell, the solubility of the shell is decreased. This corresponds to an increase in the time required for the capsule to dissolve and release its fill. In extreme cases, the capsules can fail to open within the body, representing a complete failure of the pharmaceutical and/or nutraceutical delivery system. Gelatin capsules that are comprised of higher molecular weight gelatin molecules are more prone to display dissolution problems when challenged with a chemical crosslinker because the initial degree of crosslink connectivity in the shell is more advanced. The effect of chemical crosslinkers on gelatin is also known to be highly pH dependent (see Ref C), Ref D), Ref E)).

Common gelatin manufacturing processes, known in the art, result in significant fractions of microgel being present in most gelatin extracts. The specific amount varies based on raw material type, pretreatment conditions and extraction conditions. To help reduce the impact of microgel in gelatin capsules, it has been shown that the inclusion of 1 to 20% of small peptides of between approximately 100 to 1000 Da into a gelatin composition can reduce the impact of chemical crosslinkers on gelatin dissolution Ref) G. This improvement occurs because as new crosslinks form within the capsule shell some of those crosslinks are formed with the small peptides rather than with the larger gelatin molecules. This limits the potential growth of the microgel molecules and helps to maintain the dissolution profile of the capsule over time.

While effective, the addition of small peptides into a gelatin composition, may not fully compensate for the crosslinker activity of several important fills in the market such as krill oil, peppermint oil, astaxanthin, metal ions (Fe, Zn, Al), saw palmetto, chondroitin sulfate, vitamin D3, salmon oil, b-complex, lutein, multivitamin pastes, oregano oil, garlic oil, other herbal extracts, etc. Peroxide and aldehyde impurities in polyethylene glycol used as plasticizers in capsule shell formations can also cause significant gelatin crosslinking. To further improve the performance of a gelatin composition, it is therefore desired to reduce the amount of microgel present.

Use of endoproteases and exoproteases to enzymatically hydrolyze gelatin has been known for decades. These proteases include but are not limited to serine proteases, cysteine proteases, threonine proteases, aspartic proteases, glutamic proteases, and metalloproteases. Large doses or prolonged exposure of gelatin or collagen to these of enzymes is used to produce collagen hydrolysate. Collagen hydrolysate is defined a gelatin or collagen that has been hydrolyzed to a point where the material can no longer form a gel and corresponds to having an approximate average molecular weight under 10 kDa. Low dose or brief exposure of gelatin or collagen to these enzymes is known to reduce the viscosity with little to no effect on gel strength Ref) H. This is because the enzymes are more effective against the larger native crosslinked gelatin molecules and the product of those enzymatic hydrolysis reaction is an increase in the number of a chain sized molecules. Thus, enzymatic treatment of collagen or gelatin can be used to reduce the amount of undesired microgel. As the enzyme dosage or exposure time is increased the degree of hydrolyzation becomes more extensive, resulting in a significant reduction of a chains sized molecules, a reduction in gel strength and a continued reduction of viscosity.

Unfortunately, the practical application of enzymes into industrial gelatin pretreatment, extraction and processing has several important limitations and challenges. Enzyme formulations used in the gelatin industry are usually not mono-compositional (being composed of a single unique enzyme) and instead are concentrated enzymatic cocktails that are comprised of several unique enzymes extracted simultaneously from various plant, animal, or microbiological sources. These enzyme cocktails are often standardized for activity with maltodextrin, which can cause undesired turbidity or Maillard browning in gelatin lots. Enzyme cocktails that are sold as fine dried powders can cause strong allergic reactions following trace powder exposure and represent a significant safety concern regarding their use in gelatin manufacturing. Enzyme cocktails suppled in liquid form often utilize significant concentrations of glycerin or sorbate as stabilizers. The stabilizers glycerin and sorbate are very hydroscopic and can make the drying of the gelatin more demanding. These residual stabilizers can also increase the hydroscopic character the dried gelatin lots leading to increased moisture uptake during storage or shipment. Controlling the extent of an enzymatic treatment can also be challenging. During the manufacture of collagen peptides, the enzymatic dosage tolerance and process control is more forgiving because as the average molecular weight is reduced under 10 kDa the number of enzymatic liable sites become highly depleted. This leads to a slowing reaction rate in the targeted viscosity or molecular weight region. During the manufacture of much high molecular weight gelatin, however the enzymatic dosage tolerance and process control are more demanding because the number of enzymatic liable sites remains high. Thus, the reaction rate in the target viscosity or molecular weight region remains high and is more sensitive to small variations in pH, temperature, cofactor concentration, salt concentrations and the molecular weight distribution of the initial gelatin composition.

### BRIEF SUMMARY OF THE INVENTION

The invention provides for a more efficient method to produce a gelatin composition with reduced amounts of undesirable microgel components for use in capsule or tablet coating applications. The invention also provides for a more efficient method to reduce the viscosity of gelatin compositions extracted from a collagen containing raw material to better match the quality and quantity demands of the gelatin marketplace. In particular, the invention provides method to increase the rate of viscosity degradation of a gelatin, comprising the steps of: providing an aqueous solution of gelatin;
combining the aqueous solution of gelatin with about 2 to about 20% reactive peptides (in terms of the weight fraction of the reactive peptides relative to the total dissolved protein content of the aqueous solution), wherein the reactive peptides comprise collagen peptides with an average molecular weight of about 100 to about 5,000 Da and/or amino acids;
adjusting a pH of the aqueous solution about 3.4 to about 8.0;
heating the aqueous solution to a temperature from about 60 to about 150° C.; and
holding the aqueous solution at said temperature for a period of time sufficient to degrade the viscosity of the solution to be in a range from about 15 to about 25 mP, when measured at a total dissolved protein concentration of 6.67% w/w, at a temperature of 60° C. and at a pH of 5.3 to 5.5.

The interaction of the reactive peptides with the gelatin composition increases the degradation rate of the viscosity and microgel components significantly and provides for a more efficient manufacturing process. Another aspect of the invention is that the reactive peptides can be either partially or fully recovered using ultrafiltration or diafiltration.

The invention also provides a gelatin composition that can be utilized to produce capsules with improved dissolution performance in the presence of crosslinking chemical agents. In particular, the invention provides a gelatin composition comprising gelatin and about 2 to about 20% reactive peptides (in terms of the weight fraction of the reactive peptides relative to the total protein content of the gelatin composition), wherein the reactive peptides comprise collagen peptides with an average molecular weight of about 100 to about 5,000 Da and/or amino acids, and wherein the gelatin composition has
a viscosity from 15 to 17 mP and a Bloom value from 55 to 171 g, or
a viscosity from 17 to 19 mP and a Bloom value from 73 to 189 g, or
a viscosity from 19 to 21 mP and a Bloom value from 91 to 205 g, or
a viscosity from 21 to 23 mP and a Bloom value from 109 to 220 g, or
a viscosity from 23 to 25 mP and a Bloom value from 124 to 233 g,
when measured at a total dissolved protein concentration of 6.67% w/w, at a temperature of 60° C. and at a pH of 5.3 to 5.5.

In the present description and claims, all percentages are weight %, unless explicitly stated otherwise.

### BRIEF DESCRIPTION OF THE SEVERAL VIEWS OF THE DRAWING(S)

FIG. 1. A plot of the viscosity versus the average molecular weight of several commercial limed bovine bone gelatin batches. The viscosity is measured at 6.67% w/w concentration and a temperature of 60° C.

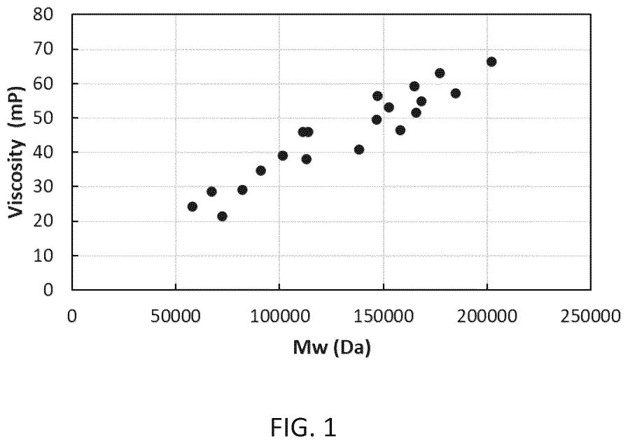

FIG. 2. A plot of the viscosity versus the fraction of microgel present in several commercial limed bovine bone gelatin batches. The viscosity is measured at 6.67% w/w concentration and a temperature of 60° C.

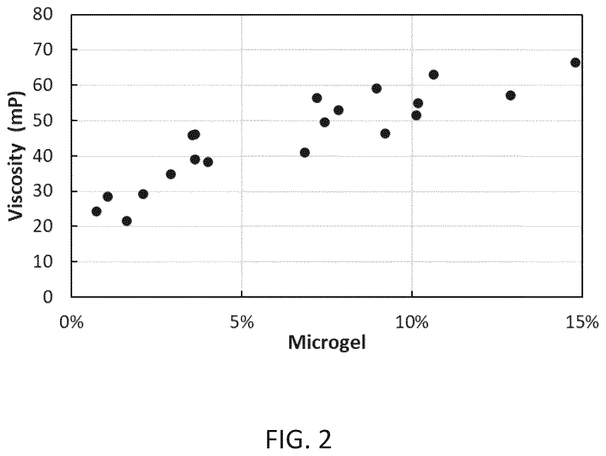

FIG. 3. Illustrative picture showing difference regions of the gelatin size exclusion chromatographs. The solid curve shows the chromatograph of a standard limed bovine bone gelatin reference. The dashed curve shows the chromatograph of reactive peptides that were composed of collagen peptides with an average molecular weight of 690 Da. The fine dashed arrows denote the eluting volume cutoffs boundaries between the different molecular regions as described in Example 3.

### DETAILED DESCRIPTION OF THE INVENTION

Aspects of the invention provide for an improved method to reduce the amount of undesirable microgel components in a gelatin composition. The inventive method may ameliorate one or more of the disadvantages of gelatin performance in capsule or tableting applications by producing gelatin compositions with a reduced microgel burden.

The invention also provides for a more efficient and faster method to reduce the viscosity of a gelatin composition. The inventive method may ameliorate one of more of the disadvantages of gelatin manufacturing when the viscosity of the gelatin extracted from a collagen containing raw material is naturally higher than the viscosity requirements of the capsule or tableting markets.

One embodiment of the invention provides a method to increase the rate of viscosity degradation of a gelatin, comprising the steps of:
providing an aqueous solution of gelatin;
combining the aqueous solution of gelatin with about 2 to about 20% reactive peptides (in terms of the weight fraction of the reactive peptides relative to the total dissolved protein content of the aqueous solution), wherein the reactive peptides comprise collagen peptides with an average molecular weight of about 100 to about 5,000 Da and/or amino acids;
adjusting a pH of the aqueous solution to from about 3.4 to about 8.0;
heating the aqueous solution to a temperature from about 60 to about 150° C.; and
holding the aqueous solution at said temperature for a period of time sufficient to degrade the viscosity of the solution to be in a range from about 15 to about 25 mP, when measured at a total dissolved protein concentration of 6.67% w/w, at a temperature of 60° C. and at a pH of 5.3 to 5.5.

The reactive mixture is then preferably cooled to under 60° C. to deactivate.

In a preferred embodiment, the aqueous solution of gelatin is combined with about 9 to about 13% reactive peptides (in terms of the weight fraction of the reactive peptides relative to the total dissolved protein content of the aqueous solution). The total dissolved protein content of the aqueous solution can be determined in accordance with the Kjeldahl method.

The pH of the combined aqueous solution is preferably adjusted to from about 4.5 to about 5.5.

Preferably, the aqueous solution is heated after pH adjustment to a temperature from about 90 to about 100° C.

In a preferred embodiment, the aqueous solution is held at said temperature for a period of time sufficient to degrade the viscosity of the gelatin to be in a range from about 17 to about 22 mP, when measured at a gelatin concentration of 6.67% w/w, at a temperature of 60° C. and at a pH of 5.3 to 5.5.

The period of time sufficient for the target viscosity degradation is dependent on various parameters, including the temperature at which the solution is held. In preferred embodiments, the aqueous solution is held at said temperature for less than about 24 h, for less than about 10 h, or for less than about 5 h.

Aspects of the invention provide for the aqueous gelatin solution to comprise a Type A gelatin, Type B gelatin, of a combination thereof. In aspects of the invention, the gelatin is derived from bovine bone, bovine hide, porcine bone, porcine skin, fish skin, chicken bone, jellyfish or a combination thereof. In other aspects of the invention the gelatin solution can be derived from single or multiple gelatin extracts or production batches. In another implementation of the invention, the gelatin solution can be made from the dissolution of a dried gelatin powder or granular. In another implementation of the invention, the gelatin solution can be comprised of a dried blended gelatin powder or dried blended granular. In yet another implementation of the invention, the gelatin solution is comprised of multiple dried blended gelatin powders or multiple dried blended gelatin granules.

In one implementation of the invention, the reactive peptides are collagen peptides that have an average molecular weight from about 100 Da to about 5,000 Da. In aspects of the invention, the collagen peptides may have an average molecular weight from about 100 Da to about 1,000 Da, from about 1,000 Da to about 3,000 Da, from about 3,000 Da to about 5,000 Da or a range defined by any two of the foregoing values. In one preferred implementation of the invention, the collagen peptides have an average molecular weight from about 400 Da to about 800 Da.

In a preferred embodiment of the invention, the collagen peptides are obtained by enzymatic hydrolysis, preferably of Type A gelatin, Type B gelatin, collagen, a collagen containing animal tissue or bone, or a combination thereof.

In another implementation of the invention, the reactive peptides further comprise peptides that are derived from hydrolyzed soy, casein, whey, or pea protein, or a combination thereof, wherein the peptides have an average molecular weight of about 100 to about 5,000 Da.

In another preferred implementation of the invention, the reactive peptides comprise amino acids, in addition to or instead of the collagen peptides. In particular, the amino acids are selected from alanine, arginine, asparagine, aspartic acid, cysteine, glutamine, glutamic acid, glycine, diglycine, histidine, isoleucine, lysine, hydroxylysine, methionine, phenylalanine, proline, hydroxyproline, serine, threonine, tryptophan, tyrosine, valine, or a combination thereof. The reactive peptides can also comprise diglycine.

In a preferred implementation of the invention, the reactive peptides are added to the aqueous gelatin solution as dried solids and dissolved. In another implementation of the invention, the reactive peptides are dissolved to form an aqueous solution of reactive peptides and are then wet blended into the aqueous gelatin solution.

The reactive peptides utilized in the inventive method are combined with the starting gelatin solution, wherein from about 2% to about 20% reactive peptides (in terms of the weight fraction of the reactive peptides relative to the total dissolved protein content of the aqueous solution) are used to produce a pre-reactive mixture. In aspects of the invention, the collagen peptides are used as reactive peptides and are present in an amount from 9 to 13% of a total dissolved protein content of the pre-reactive mixture. For example, the collagen peptides may be added in the amount of 2%, 3%, 4%, 5%, 6%, 7%, 8%, 9%, 10%, 11%, 12%, 13%, 14%, 15%, 16%, 17%, 18%, 19%, 20%, or a range defined by any two of the foregoing values, of a total dissolved protein content of the reactive mixture.

In a preferred aspect of the invention, the pH of the initial aqueous gelatin solution is adjusted by passing the solution through a series of cationic and anionic ion exchange resins. In another aspect of the invention, the pH of the initial aqueous gelatin solution is adjusted through the addition of hydrochloric acid, sulfuric acid, phosphoric acid, nitric acid, citric acid, acetic acid, sodium monobasic phosphate, potassium monobasic phosphate or by a mixture of chemicals comprising any combination of the foregoing compounds. In aspects of the invention, the initial pH of the initial aqueous gelatin solution is adjusted through the addition of sodium hydroxide, calcium hydroxide, magnesium hydroxide, potassium hydroxide, dicalcium phosphate, dicalcium phosphate dihydrate, sodium dibasic phosphate, potassium dibasic phosphate, calcium citrate, sodium citrate, calcium acetate, sodium acetate or by a mixture of chemicals comprising any combination of the foregoing compounds.

In the method of the invention, the pH of the pre-reactive mixture is adjusted to from about 3.4 to about 8.0, with a preferred range of from about 4.5 to about 5.5. In aspects of the invention, the pH of the pre-reactive mixture is adjusted through the addition of hydrochloric acid, sulfuric acid, phosphoric acid, nitric acid, citric acid, acetic acid, sodium monobasic phosphate, potassium monobasic phosphate or by a mixture of chemicals comprising any combination of the foregoing compounds. In aspects of the invention the pH of the pre-reactive mixture is adjusted through the addition of sodium hydroxide, calcium hydroxide, magnesium hydroxide, potassium hydroxide, dicalcium phosphate, dicalcium phosphate dihydrate, sodium dibasic phosphate, potassium dibasic phosphate, calcium citrate, sodium citrate, calcium acetate, sodium acetate or by a mixture of chemicals comprising any combination of the foregoing compounds.

In the method of the invention, the reactive mixture is heated to from about 60 to about 150° C., and held for preferably up to 24 hours. In a preferred aspect of the invention, the reactive mixture is held between about 90 and about 100° C. and is held for ≤4 hours. In another implementation of the invention, the reactive mixture is held between about 80 and about 90° C. for ≤8 hours. In another implementation of the invention, the reactive mixture is held between about 100 and about 110° C. for ≤2 hours. In another implementation of the invention, the reactive mixture is held between about 110 and about 120° C. for ≤60 minutes. In another implementation of the invention, the reactive mixture is held between about 120 and about 130° C. for ≤30 minutes. In another implementation of the invention, the reactive mixture is held between about 130 and 140° C. for ≤15 minutes. In another implementation of the invention, the reactive mixture is held between about 140 and about 150° C. for ≤7 minutes.

According to another preferred aspect of the invention, the method further comprises the step of at least partially removing the reactive peptides from the reactive mixture after the thermal treatment using ultrafiltration, diafiltration, or a combination thereof. In another aspect of the invention, the at least partially removed reactive peptides can be recovered from the ultrafiltration or diafiltration permeate, concentrated by nanofiltration, reverse osmosis, evaporation or a combination thereof. The recovered reactive peptides can then be reused in the inventive method.

According to another aspect of the invention, a gelatin composition is provided that can be utilized to produce capsules with improved dissolution performance in the presence of chemical crosslinking agents.

One embodiment of the invention provides a gelatin composition comprising gelatin and about 2 to about 20% reactive peptides (in terms of the weight fraction of the reactive peptides relative to the total protein content of the gelatin composition), wherein the reactive peptides comprise collagen peptides with an average molecular weight of about 100 to about 5,000 Da and/or amino acids, and wherein the gelatin composition has
a viscosity from 15 to 17 mP and a Bloom value from 55 to 171 g, or
a viscosity from 17 to 19 mP and a Bloom value from 73 to 189 g, or
a viscosity from 19 to 21 mP and a Bloom value from 91 to 205 g, or
a viscosity from 21 to 23 mP and a Bloom value from 109 to 220 g, or
a viscosity from 23 to 25 mP and a Bloom value from 124 to 233 g,
when measured at a total dissolved protein concentration of 6.67% w/w, at a temperature of 60° C. and at a pH of 5.3 to 5.5.

In a preferred embodiment of the invention, the gelatin composition comprises a microgel fraction about 0.8% or less of the total protein content of the gelatin composition.

The gelatin composition preferably comprises about 9 to about 13% reactive peptides (in terms of the weight fraction of the reactive peptides relative to the total protein content of the gelatin composition).

The pH of the gelatin composition of the invention is preferably from 5.3 to 6.0.

In preferred embodiments of the invention, the collagen peptides are derived from Type A gelatin, Type B gelatin, collagen, collagen containing animal tissues, collagen containing animal bones, or a combination thereof.

In another preferred embodiment of the invention, the collagen peptides are enzymatically hydrolyzed collagen peptides.

The collagen peptides preferably have an average molecular weight of about 400 to 800 Da.

In another aspect of the invention, the reactive peptides further comprise peptides that are derived from hydrolyzed soy, casein, whey, or pea protein, or a combination thereof, and wherein the peptides have an average molecular weight of about 100 to about 5,000 Da. In another preferred implementation of the invention, the amino acids are selected from alanine, arginine, asparagine, aspartic acid, cysteine, glutamine, glutamic acid, glycine, histidine, isoleucine, lysine, hydroxylysine, methionine, phenylalanine, proline, hydroxyproline, serine, threonine, tryptophan, tyrosine, valine, or a combination thereof, and/or wherein the reactive peptides comprise diglycine.

The gelatin in the gelatin composition of the invention may be obtained from bovine bone, bovine hide, porcine bone, porcine skin, fish skin, chicken bone, jellyfish or a combination thereof.

In another aspect of the invention, the gelatin is either Type A gelatin, Type B gelatin, or a combination thereof.

Preferably, the gelation composition is in the form of a granule or powder obtained by drying and milling.

In a preferred embodiment of the gelatin composition of the invention, the gelatin composition comprises a gelatin obtained by the method according to the invention.

Further advantages and preferred embodiments of the inventive gelatin composition have already been described in connection with the inventive method.

Another aspect of the invention relates to the use of the gelatin composition of the invention for the manufacture of gelatin capsules. Preferably, the gelatin capsules are hard capsules, soft capsules or microcapsules. As described above, such capsules have an improved dissolution performance in the presence of crosslinking chemical agents.

The invention is described further by way of the following Examples, which are not to be construed as limiting the scope of the invention in any way.

### Example 1

This example demonstrates how to prepare reactive peptides comprised of a collagen peptide composition. An approximately 35% w/w solution was prepared by dissolving 1000 g of dried pigskin gelatin (Bloom of 260 g and a 6.67%/60° C. viscosity of 37.0 mP) into 1500 g of water. The pH of the solution was adjusted to between 5.0-5.1 with sulfuric acid and the temperature was held at between 58-62° C. Approximately 0.08 g of sodium metabisulfite and 17.5 g of LIQUIPANOL® T200 supplied from Enzyme Development Corporation were added to the solution. The mixture was held for 24-36 hours at 60° C. The solution was combined with 1 mL of about 30% hydrogen peroxide, heated to 95° C. and held at 95° C. for 120 minutes. The solution was then cooled to room temperature and filtered using diatomaceous earth. Size exclusion chromatograph analysis determined the average molecular weight of the collagen peptide was 590 Da. The method can be equally applied to both Type A or Type B gelatins. One skilled in the art will recognize that the average molecular weight can be altered by changing the reaction time, the reaction pH, the reaction temperature, enzyme concentration or type of enzyme used.

### Example 2

This example demonstrates the ability of the reactive peptides to increase the rate of viscosity degradation and that the efficacy of reactive peptides increases as their average molecular weight is decreased.

A dried limed bovine bone gelatin (Bloom of 273 g and a 6.67%/60° C. viscosity of 45.9 mP) was dissolved into 55-60° C. water to produce an 20% w/w aqueous gelatin solution. The gelatin solution was then combined with 11% reactive peptides which were comprised of collagen peptides compositions produced from limed bovine bone gelatin. The solution had its pH adjusted to 5.4 and was then placed in a water bath of 85° C. (covered with a watch glass) for 6 hours. The solution was allowed to cool to 55-60° C. Described above process was repeated 3 times, using collagen peptides compositions of different average molecular weights. The solutions were then diluted to approximately 10%, the pH was readjusted with sulfuric acid or sodium hydroxide (if needed) to 5.4, then the solutions were further diluted to 6.67% and had their viscosities measured with a gravity pipette at 60° C.

A second series of control 20% w/w gelatin aqueous solutions were prepared using the same initial dried gelatin lot. Each solution had its pH adjusted to 5.4 and was placed in a water bath of 85° C. (covered) for 6 hours. The solutions were then allowed to cool to 55-60° C. To compare the viscosity of control examples to the inventive examples, the control solutions were then combined with 11% reactive peptides which were comprised of the same collagen peptides compositions utilized in the examples of the inventive method. The solutions were then diluted to approximately 10% w/w, the pH was readjusted with sulfuric acid or sodium hydroxide (if needed) to 5.4, then the solutions were further diluted to 6.67% w/w and had their viscosities measured with a gravity pipette at 60° C.

Table A demonstrates the inventive method produces a larger gelatin viscosity degradation given a fixed reaction time. The viscosity is measured at 6.67% w/w and 60° C. The presence of the reactive peptides significantly increased the rate of viscosity degradation compared to the control case when the same reactive peptides were not added until after the 85° C. thermal treatment. The viscosity degradation was the largest when the reactive peptides where collagen peptide compositions had an average molecular weight of approximately 650 Da.

TABLE A

| | Invention Method | Control Example |
|---|---|---|
| | Viscosity | Viscosity |
| | (6.67%/60° C.) | (6.67%/60° C.) |
| Collagen peptides (~650 Da) | 19.4 mP | 25.0 mP |
| Collagen peptides (~2000 Da) | 22.1 mP | 25.0 mP |
| Collagen peptides (~4900 Da) | 25.6 mP | 27.2 mP |

### Example 3

This example demonstrates the ability of the reactive peptides to increase the rate of viscosity and microgel degradation of a Type B gelatin and that the efficacy of reactive peptides is pH dependent.

A dried limed bovine bone gelatin (Bloom of 280 g and a 6.67%/60° C. viscosity of 41.9 mP) was dissolved into 55-60° C. water to produce an 20% w/w aqueous gelatin solution. This solution was then divided into 2 fractions. The first fraction of this initial gelatin solution was used to demonstrate the inventive method. This fraction was combined with 11% reactive peptides which were comprised of a dried collagen peptide composition, produced from a limed bovine bone gelatin, and had an average molecular weight of 650 Da. Once the dried collagen peptides were fully dissolved, the pH of the solution was adjusted to one of the targeted setpoints given below using either sulfuric acid or sodium hydroxide. The solution (covered) was then placed into an 85° C. water bath. Throughout the thermal hold period, samples of the gelatin solution were extracted for analysis at various timepoints. Once removed from the 85° C. solution, the timepoint samples were cooled to between 55-60° C. The different timepoint samples were then diluted to roughly a 10% w/w concentration. The pH of each specific timepoint solution was adjusted to 5.4 using either sulfuric acid or sodium hydroxide. The timepoint samples were then further diluted to 6.67% w/w and their viscosity measured with a gravity pipette at 60° C. These measured viscosities for the inventive method are provided in Table B and are reported in units of mP.

The second fraction of the initial gelatin aqueous solution was used a control example and was processed alongside of the inventive example. This fraction had its pH adjusted to the same setpoint as the inventive fraction using either sulfuric acid or sodium hydroxide. The solution (covered) was then placed into the same 85° C. water bath as used with the inventive method fraction. Throughout the thermal hold period, samples of the control gelatin solution were extracted for analysis at various timepoints. Once removed from the 85° C. solution, the timepoint samples were cooled to between 55-60° C. To compare the inventive method and control example, the same dried collagen peptide composition used in the inventive method was added produce a solution with 11% concentration of reactive peptides, with the amount required being calculated from the measured mass of the timepoint sample and its concentration. Once added the dried collagen peptides were allowed to fully dissolve. The different timepoint samples were then diluted to roughly a 10% w/w concentration. The pH of the each specific timepoint solution was then adjusted to 5.4 using either sulfuric acid or sodium hydroxide. The samples were then further diluted to 6.67% w/w and their viscosity measured with a gravity pipette at 60° C. These measured viscosities for the control example are provided in Table C and are reported in units of mP.

The pH setpoints used were 6.6, 5.8, 5.4, 5.0, 4.6, 4.4, 4.0 or 3.6.

TABLE B

| Time | pH setpoints | | | | | | | |
|---|---|---|---|---|---|---|---|---|
| (hrs) | 3.6 | 4.0 | 4.4 | 4.6 | 5.0 | 5.4 | 5.8 | 6.6 |
| 0.0 | 33.48 | 34.07 | 36.40 | 33.48 | 33.48 | 36.40 | 36.11 | 36.11 |
| 0.5 | 23.04 | 26.21 | 29.60 | 25.59 | | | | |
| 1.0 | 17.76 | 21.75 | 24.32 | | 28.99 | 32.60 | 32.60 | 34.07 |
| 1.5 | 14.66 | 16.74 | | | | | | |
| 2.0 | 13.24 | 15.01 | 20.44 | 21.75 | 24.64 | 28.68 | 30.51 | 32.00 |
| 3.0 | 10.71 | 12.89 | 17.76 | 18.44 | 21.10 | 25.90 | 28.38 | |
| 3.5 | | | | | | | | 29.60 |
| 3.75 | | | | | 20.11 | | | |
| 4.0 | | 11.07 | 15.71 | 17.08 | | | | |
| 4.5 | 8.47 | | | | | | | |
| 5.0 | | | | | 18.44 | | | |
| 5.25 | | | | | | 21.75 | | |
| 5.5 | | | | 14.31 | | | | |
| 6.0 | | | | | | | 23.69 | 26.52 |
| 22.0 | | | 7.32 | | 9.59 | | | |
| 22.5 | 4.14 | 4.97 | | | | | | |
| 23.0 | | | | 8.09 | 9.22 | 11.44 | 13.24 | 14.66 |
| 24.0 | | | | | | 11.07 | | |
| 25.0 | | | | 7.32 | | | 12.89 | 14.31 |
| 26.5 | | 4.97 | | | | | | |
| 27.25 | | | 6.55 | | | | | |

TABLE C

| Time | pH setpoints | | | | | | | |
|---|---|---|---|---|---|---|---|---|
| (hrs) | 3.6 | 4.0 | 4.4 | 4.6 | 5.0 | 5.4 | 5.8 | 6.6 |
| 0.0 | 34.07 | 34.36 | 35.24 | 34.07 | 33.48 | 36.11 | 35.82 | 37.83 |
| 0.5 | 24.00 | 27.15 | 31.11 | | | | | |
| 1.0 | 19.44 | 22.40 | 27.76 | 29.60 | 32.00 | 34.95 | 34.95 | 36.40 |
| 1.5 | 16.05 | 18.44 | | | | | | |
| 2.0 | 14.66 | 16.74 | 24.00 | 24.96 | 28.99 | 32.00 | 32.48 | 33.48 |
| 3.0 | 11.44 | 14.31 | 19.44 | 22.40 | 26.21 | 31.11 | 32.60 | |
| 3.5 | | | | | | | | 32.30 |
| 3.75 | | | | | 25.59 | | | |
| 4.0 | | 12.53 | 18.44 | 20.77 | | | | |
| 4.5 | 9.22 | | | | | | | |
| 5.0 | | | | | 24.00 | | | |
| 5.25 | | | | | | 27.46 | | |
| 5.5 | | | | 18.44 | | | | |
| 6.0 | | | | | | | 29.29 | 29.29 |
| 22.0 | | | 9.22 | | 13.24 | | | |
| 22.5 | 4.55 | 5.77 | | | | | | |
| 23.0 | | | | 11.07 | 12.89 | 16.74 | 18.10 | 19.11 |
| 24.0 | | | | | | 16.40 | | |
| 25.0 | | | | 9.97 | | | 17.42 | 18.44 |
| 26.5 | | 5.77 | | | | | | |
| 27.25 | | | 8.47 | | | | | |

The viscosity degradation for each condition was found to be well described by the following empirical equation,

$$\frac{1}{η} = {x}_{0} + {x}_{1} exp ( - {x}_{2} t ) \qquad ( 1 )$$

where η is the gelatin viscosity at 6.67% CDG and 60° C., tis the thermal hold time and x₀, x₁, and x₂ are least-squares fitted parameters. Using standard regression techniques available in the current art it is possible to determine a set of x₀, x₁, and x₂ for each pH and allows for the interpolation of time required to reach a targeted viscosity.

TABLE D shows the interpolated times required to obtain 3 different targeted viscosities using both the inventive method and the control example. The viscosity of the samples utilizing the present inventive process degraded between 13% to 138% faster than the control example using only a thermal treatment.

TABLE D

| | Target = 25 mP | | Target = 20 mP | | Target = 15 mP | |
|---|---|---|---|---|---|---|
| | Invention | Control | Invention | Control | Invention | Control |
| | Method | Method | Method | Method | Method | Method |
| pH | Time (hrs) | Time (hrs) | Time (hrs) | Time (hrs) | Time (hrs) | Time (hrs) |
| 3.6 | 0.3 | 0.5 | 0.8 | 0.9 | 1.6 | 1.8 |
| 4.0 | 0.6 | 0.7 | 1.1 | 1.4 | 2.2 | 2.7 |
| 4.4 | 1.1 | 1.6 | 2.3 | 3.2 | 4.5 | 6.3 |
| 4.6 | 1.2 | 2.1 | 2.5 | 4.5 | 5.2 | 9.3 |
| 5.0 | 1.9 | 4.2 | 3.9 | 8.5 | 7.8 | 16.8 |
| 5.4 | 3.5 | 7.7 | 6.5 | 14.8 | 12.6 | >30 |
| 5.8 | 5.0 | 10.4 | 9.5 | 18.7 | 18.1 | >30 |
| 6.6 | 7.1 | 10.9 | 12.8 | 20.5 | 22.6 | >30 |

Samples of each solution were then diluted further to about 0.1%. These samples were saved in a refrigerator at 4-8° C. temporarily until all the timepoints for a particular pH degradation run were collected. The retained samples were then analyzed using size exclusion chromatography.

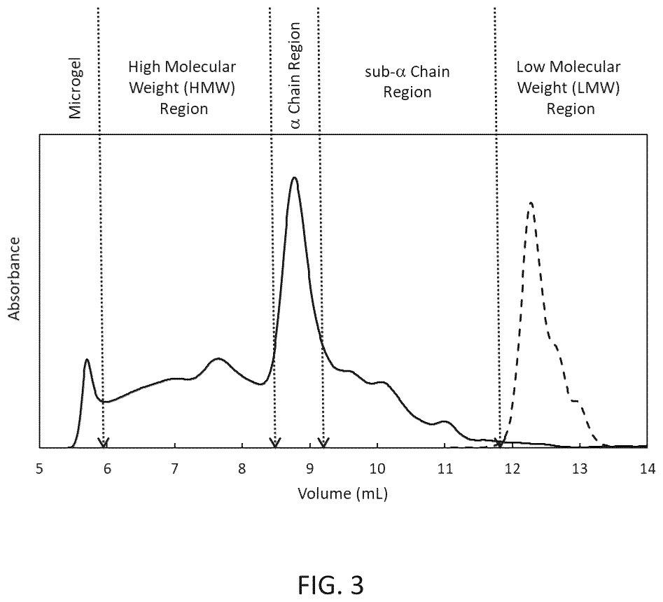

The size exclusion chromatographic method utilized a mobile phase comprised of 8.67 mM sodium dodecyl sulfate, 10 mM sodium phosphate, 100 mM sodium sulfate and was pH adjusted to 5.2 using NaOH. The column used was the TOSOH TSK-GEL G4000SWx1 (7.8 mm×30 mm). The flow rate was 0.578 mL/min, the injection volume was 50 μL and the time of each run was 30 minutes. The chromatograph was determined by measuring the amount of absorbance at 214 nm. Each chromatograph was then normalized. The normalized chromatographs were divided into 5 different regions and the fractional mass of protein in each region was determined through numerical integration (trapezoidal method). The 5 different regions were defined as the microgel, high molecular weight (HMW) region, a-chain region, sub-a chain region and the low molecular weight (LMW) region. FIG. 3 provides a graphical representation of these molecular weight regions as overlayed onto the resulting chromatographs.

The center of the α peak in terms of mL of eluted solution was determined for each chromatographic sample set by using a reference standard gelatin where the α peak is well defined. The lower and upper limits of the α chain region was defined as ±0.35 mL from center of this easily identifiable chromatographic peak. The microgel peak in the reference standard gelatin is also well defined. The microgel region was set as the amount of eluted material before the first minimum after the microgel peak. All material eluting after the microgel region and before the α region was classified as the HMW region. A second reference solution comprising only the collagen peptide composition utilized was also tested. The start location of the collagen peptide peak is also clearly definable in the chromatographs (usually around 11.9-12.1 mL). All material eluting after the start of the collagen peptide peak was classified as inside the LMW region. Material eluting between the end of the α chain region and the start of the LMW region was classified as the sub «-alpha region.

TABLE E provides the molecular weight distributions produced by the inventive method at a pH of 3.6 and demonstrates an increased rate of microgel degradation compared the control example.

TABLE F provides the molecular weight distributions produced by the inventive method at a pH of 4.0 and demonstrates an increased rate of microgel degradation compared the control example.

TABLE G provides the molecular weight distributions produced by the inventive method at a pH of 4.4 and demonstrates an increased rate of microgel degradation compared the control example.

TABLE H provides the molecular weight distributions produced by the inventive method at a pH of 4.6 and demonstrates an increased rate of microgel degradation compared the control example.

TABLE I provides the molecular weight distributions produced by the inventive method at a pH of 5.0 and demonstrates an increased rate of microgel degradation compared the control example.

TABLE J provides the molecular weight distributions produced by the inventive method at a pH of 5.4 and demonstrates an increased rate of microgel degradation compared the control example.

TABLE K provides the molecular weight distributions produced by the inventive method at a pH of 5.8 and demonstrates an increased rate of microgel degradation compared the control example.

TABLE L provides the molecular weight distributions produced by the inventive method at a pH of 6.6 and demonstrates an increased rate of microgel degradation compared the control example.

TABLE E

| Process | Reaction | | | | sub-α | |
|---|---|---|---|---|---|---|
| (pH held at 3.6) | Time (hrs) | Microgel | HMW | α peak | peak | LMW |
| Present Invention | 0.0 | 2.7% | 34.7% | 25.7% | 26.8% | 10.0% |
| | 0.5 | 0.5% | 23.5% | 19.5% | 44.8% | 11.5% |
| | 1.0 | 0.1% | 9.2% | 11.5% | 64.2% | 15.0% |
| | 1.5 | 0.1% | 9.2% | 11.5% | 64.2% | 15.0% |
| | 2.0 | 0.1% | 5.8% | 8.8% | 68.2% | 17.0% |
| | 3.0 | 0.1% | 2.3% | 5.3% | 71.1% | 21.1% |
| | 4.5 | 0.0% | 0.7% | 2.5% | 69.8% | 26.9% |
| | 22.5 | 0.1% | 0.1% | 0.2% | 44.5% | 55.1% |
| Control | 0.0 | 2.6% | 34.5% | 25.5% | 26.9% | 10.4% |
| | 0.5 | 0.6% | 24.9% | 19.4% | 43.7% | 11.3% |
| | 1.0 | 0.2% | 16.9% | 15.3% | 54.6% | 12.9% |
| | 1.5 | 0.1% | 11.3% | 12.3% | 61.7% | 14.6% |
| | 2.0 | 0.1% | 7.6% | 9.8% | 65.6% | 16.6% |
| | 3.0 | 0.1% | 3.7% | 6.6% | 69.5% | 19.9% |
| | 4.5 | 0.1% | 1.4% | 3.8% | 69.4% | 25.1% |
| | 22.5 | 0.0% | 0.0% | 0.1% | 49.2% | 50.7% |

TABLE F

| Process | Reaction | | | | sub-α | |
|---|---|---|---|---|---|---|
| (pH held at 4.0) | Time (hrs) | Microgel | HMW | α peak | peak | LMW |
| Present Invention | 0.0 | 2.7% | 34.7% | 25.7% | 26.5% | 10.3% |
| | 0.5 | 0.8% | 27.0% | 20.9% | 40.0% | 11.1% |
| | 1.0 | 0.3% | 20.0% | 17.3% | 50.1% | 12.1% |
| | 1.5 | 0.1% | 14.4% | 14.3% | 57.6% | 13.4% |
| | 2.0 | 0.1% | 10.9% | 12.3% | 62.1% | 14.6% |
| | 3.0 | 0.1% | 6.0% | 8.8% | 67.7% | 17.2% |
| | 4.0 | 0.1% | 3.7% | 6.8% | 70.0% | 19.3% |
| | 22.5 | 0.0% | 0.0% | 0.2% | 51.7% | 47.9% |
| | 26.5 | 0.0% | 0.0% | 0.0% | 42.5% | 57.3% |
| Control | 0.0 | 2.6% | 34.8% | 25.6% | 26.9% | 10.0% |
| | 0.5 | 1.0% | 28.3% | 20.5% | 39.3% | 10.8% |
| | 1.0 | 0.4% | 22.4% | 17.0% | 47.6% | 12.4% |
| | 1.5 | 0.2% | 17.3% | 14.5% | 54.5% | 13.3% |
| | 2.0 | 0.1% | 13.9% | 12.9% | 58.2% | 14.7% |
| | 3.0 | 0.1% | 8.8% | 10.1% | 64.1% | 16.7% |
| | 4.0 | 0.1% | 6.3% | 8.5% | 66.3% | 18.8% |
| | 22.5 | 0.0% | 0.1% | 0.9% | 58.3% | 40.5% |
| | 26.5 | 0.0% | 0.1% | 0.7% | 56.3% | 42.9% |

TABLE G

| Process | Reaction | | | | sub-α | |
|---|---|---|---|---|---|---|
| (pH held at 4.4) | Time (hrs) | Microgel | HMW | α peak | peak | LMW |
| Present Invention | 0.0 | 2.6% | 34.8% | 25.7% | 26.3% | 10.3% |
| | 0.5 | 1.3% | 30.5% | 22.6% | 34.7% | 10.7% |
| | 1.0 | 0.6% | 25.8% | 19.8% | 42.3% | 11.3% |
| | 2.0 | 0.2% | 18.4% | 15.9% | 52.7% | 12.5% |
| | 3.0 | 0.1% | 13.1% | 13.2% | 59.4% | 13.9% |
| | 4.0 | 0.1% | 10.1% | 11.5% | 63.1% | 15.1% |
| | 22.0 | 0.1% | 0.3% | 1.5% | 64.7% | 33.3% |
| | 27.75 | 0.0% | 0.1% | 1.0% | 61.5% | 37.2% |
| Control | 0.0 | 2.6% | 34.9% | 25.8% | 26.6% | 9.9% |
| | 0.5 | 1.7% | 32.3% | 21.8% | 33.6% | 10.3% |
| | 1.0 | 1.1% | 29.3% | 18.9% | 39.2% | 11.3% |
| | 2.0 | 0.5% | 24.2% | 15.8% | 46.9% | 12.4% |
| | 3.0 | 0.3% | 20.0% | 13.9% | 51.8% | 13.9% |
| | 4.0 | 0.2% | 17.3% | 13.0% | 55.2% | 14.2% |
| | 22.0 | 0.1% | 2.0% | 4.6% | 65.1% | 28.1% |
| | 27.75 | 0.1% | 1.3% | 3.5% | 64.1% | 30.8% |

TABLE H

| Process | Reaction | | | | | |
|---|---|---|---|---|---|---|
| (pH held | Time | | | α | sub-α | |
| at 4.6) | (hrs) | Microgel | HMW | peak | peak | LMW |
| Present | 0.0 | 2.6% | 35.2% | 25.5% | 26.5% | 10.2% |
| Invention | 1.0 | 0.7% | 26.6% | 20.1% | 41.1% | 11.4% |
| | 2.0 | 0.2% | 19.7% | 16.5% | 50.7% | 12.7% |
| | 3.0 | 0.1% | 15.1% | 14.1% | 56.6% | 13.9% |
| | 4.0 | 0.1% | 11.9% | 12.4% | 60.4% | 15.1% |
| | 5.5 | 0.1% | 8.3% | 10.4% | 64.1% | 17.0% |
| | 23.0 | 0.1% | 0.5% | 2.1% | 64.0% | 33.2% |
| | 25.0 | 0.1% | 0.4% | 1.8% | 62.9% | 34.8% |
| Control | 0.0 | 2.6% | 35.3% | 25.6% | 26.5% | 9.9% |
| | 1.0 | 1.3% | 31.0% | 19.3% | 37.0% | 11.2% |
| | 2.0 | 0.6% | 26.5% | 16.2% | 44.1% | 12.4% |
| | 3.0 | 0.4% | 23.7% | 14.7% | 47.9% | 13.2% |
| | 4.0 | 0.3% | 21.0% | 13.6% | 50.9% | 14.1% |
| | 5.5 | 0.2% | 17.0% | 12.4% | 54.7% | 15.6% |
| | 23.0 | 0.1% | 3.4% | 5.9% | 62.9% | 27.5% |
| | 25.0 | 0.1% | 2.9% | 5.4% | 62.7% | 28.6% |

TABLE I

| Process | Reaction | | | | | |
|---|---|---|---|---|---|---|
| (pH held | Time | | | α | sub-α | |
| at 5.0) | (hrs) | Microgel | HMW | peak | peak | LMW |
| Present | 0.0 | 2.4% | 35.7% | 25.9% | 25.6% | 10.5% |
| Invention | 1.0 | 0.9% | 30.2% | 22.4% | 35.2% | 11.1% |
| | 2.0 | 0.4% | 25.0% | 19.3% | 43.3% | 12.0% |
| | 3.0 | 0.2% | 21.0% | 17.2% | 48.8% | 12.8% |
| | 4.0 | 0.1% | 18.1% | 15.9% | 52.3% | 13.5% |
| | 5.0 | 0.1% | 15.5% | 14.6% | 55.5% | 14.2% |
| | 22.0 | 0.1% | 1.7% | 4.6% | 67.3% | 26.3% |
| | 23.0 | 0.1% | 1.6% | 4.3% | 67.2% | 26.9% |
| Control | 0.0 | 2.4% | 35.6% | 25.6% | 26.1% | 10.4% |
| | 1.0 | 1.8% | 34.5% | 21.2% | 31.6% | 10.8% |
| | 2.0 | 1.3% | 32.7% | 18.5% | 35.9% | 11.6% |
| | 3.0 | 1.0% | 30.7% | 17.0% | 39.0% | 12.2% |
| | 4.0 | 0.8% | 29.3% | 16.0% | 41.2% | 12.7% |
| | 5.0 | 0.6% | 27.7% | 15.3% | 43.4% | 13.0% |
| | 22.0 | 0.1% | 10.4% | 10.4% | 58.6% | 20.5% |
| | 23.0 | 0.1% | 9.9% | 10.2% | 59.0% | 20.7% |

TABLE J

| Process | Reaction | | | | | |
|---|---|---|---|---|---|---|
| (pH held | Time | | | α | sub-α | |
| at 5.4) | (hrs) | Microgel | HMW | peak | peak | LMW |
| Present | 0.0 | 2.9% | 35.8% | 25.4% | 25.3% | 10.5% |
| Invention | 1.0 | 1.6% | 32.7% | 23.5% | 31.2% | 10.9% |
| | 2.0 | 1.1% | 30.1% | 21.1% | 36.3% | 11.4% |
| | 3.0 | 0.6% | 27.1% | 19.8% | 40.6% | 11.8% |
| | 5.25 | 0.2% | 20.6% | 17.3% | 48.4% | 13.4% |
| | 23.0 | 0.0% | 3.6% | 7.0% | 66.8% | 22.6% |
| | 24.0 | 0.0% | 3.2% | 6.7% | 66.8% | 23.2% |
| Control | 0.0 | 2.9% | 36.4% | 21.8% | 28.3% | 10.6% |
| | 1.0 | 2.6% | 35.8% | 25.6% | 26.0% | 9.9% |
| | 2.0 | 2.2% | 35.1% | 20.0% | 31.5% | 11.1% |
| | 3.0 | 1.8% | 34.0% | 18.8% | 33.8% | 11.6% |
| | 5.25 | 1.1% | 31.3% | 16.8% | 38.4% | 12.3% |
| | 23.0 | 0.1% | 15.0% | 11.9% | 54.3% | 18.7% |
| | 24.0 | 0.1% | 14.4% | 11.8% | 55.2% | 18.4% |

TABLE K

| Process | Reaction | | | | | |
|---|---|---|---|---|---|---|
| (pH held | Time | | | α | sub-α | |
| at 5.8) | (hrs) | Microgel | HMW | peak | peak | LMW |
| Present | 0.0 | 2.4% | 36.0% | 25.2% | 26.0% | 10.4% |
| Invention | 1.0 | 1.6% | 33.4% | 24.3% | 29.9% | 10.7% |
| | 2.0 | 1.0% | 30.9% | 23.0% | 33.9% | 11.1% |
| | 3.0 | 0.6% | 28.3% | 22.1% | 37.4% | 11.6% |
| | 6.0 | 0.2% | 21.8% | 19.0% | 46.1% | 12.9% |
| | 23.0 | 0.0% | 5.4% | 9.2% | 64.4% | 20.9% |
| | 25.0 | 0.0% | 4.7% | 8.6% | 65.2% | 21.5% |
| Control | 0.0 | 2.4% | 35.9% | 25.6% | 26.2% | 9.8% |
| | 1.0 | 2.0% | 35.4% | 23.7% | 28.3% | 10.5% |
| | 2.0 | 1.7% | 34.6% | 22.1% | 30.4% | 11.1% |
| | 3.0 | 1.5% | 34.0% | 21.0% | 32.8% | 10.8% |
| | 6.0 | 0.8% | 31.0% | 18.1% | 37.7% | 12.3% |
| | 23.0 | 0.0% | 16.8% | 13.1% | 52.5% | 17.5% |
| | 25.0 | 0.0% | 16.0% | 12.5% | 53.4% | 17.9% |

TABLE L

| Process | Reaction | | | | | |
|---|---|---|---|---|---|---|
| (pH held | Time | | | α | sub-α | |
| at 6.6) | (hrs) | Microgel | HMW | peak | peak | LMW |
| Present | 0.0 | 2.5% | 35.5% | 25.3% | 26.4% | 10.2% |
| Invention | 1.0 | 1.8% | 33.9% | 24.6% | 29.0% | 10.5% |
| | 2.0 | 1.2% | 31.4% | 24.0% | 32.5% | 10.9% |
| | 3.5 | 0.7% | 29.1% | 23.0% | 35.8% | 11.3% |
| | 6.0 | 0.3% | 24.4% | 21.3% | 41.8% | 12.1% |
| | 23.0 | 0.0% | 7.9% | 11.5% | 62.2% | 18.3% |
| | 25.0 | 0.0% | 7.0% | 10.8% | 63.2% | 19.0% |
| Control | 0.0 | 2.5% | 35.5% | 25.3% | 26.5% | 10.0% |
| | 1.0 | 2.1% | 34.9% | 24.8% | 27.8% | 10.3% |
| | 2.0 | 1.5% | 33.5% | 24.0% | 30.2% | 10.7% |
| | 3.5 | 1.1% | 32.1% | 23.1% | 32.5% | 11.0% |
| | 6.0 | 0.7% | 29.5% | 21.2% | 36.7% | 11.8% |
| | 23.0 | 0.0% | 16.3% | 14.6% | 52.8% | 16.2% |
| | 25.0 | 0.0% | 15.4% | 14.0% | 53.7% | 16.8% |

Review of the size exclusion chromatographs finds that the inventive process reduced the microgel and HMW fraction of the gelatin at a significantly higher rate than from thermal treatment alone. Using a process pH of 4.4 and 2.0 hours of reaction time, reduced the microgel content from 2.6% to 0.2% using the present invention, while a thermal process only reduced the microgel content from 2.6% to only 0.5%. Using a process pH of 5.0 and 3.0 hours of reaction time, reduced the microgel content from 2.4% to 0.2% using the present invention, while a thermal process only reduced the microgel content from 2.4% to only 1.0%. Using a process pH of 5.8 and 3.0 hours of reaction time, reduced the microgel content from 2.4% to 0.7% using the present invention, while a thermal process only reduced the microgel content from 2.4% to only 1.5%. Using a process pH of 6.6 and 6.0 hours of reaction time, reduced the microgel content from 2.5% to 0.3% using the present invention, while a thermal process only reduced the microgel content from 2.5% to only 0.7%.

### Example 4

This example demonstrates the ability of the reactive peptides to increase the rate of viscosity and microgel degradation of a Type A gelatin.

A dried acid processed pig skin gelatin (Bloom of 275 g and a 6.67%/60° C. viscosity of 41.8 mP) was dissolved into 55-60° C. water to produce an 20% w/w aqueous gelatin solution. This solution was then divided into 2 fractions. The first fraction of this initial gelatin solution was used to demonstrate the inventive method. This fraction was combined with 11% reactive peptides which were comprised of a dried collagen peptide composition, produced from an acid processed pig skin gelatin, and had an average molecular weight of 590 Da. Once the dried collagen peptides were fully dissolved, the pH of the solution was adjusted to 5.4 using sodium hydroxide. The solution (covered) was then placed into an 85° C. water bath. Throughout the thermal hold period, samples of the gelatin solution were extracted for analysis at various timepoints. Once removed from the 85° C. solution, the timepoint samples were cooled to between 55-60° C. The solutions were then diluted to approximately 10% w/w, the pH was readjusted with sulfuric acid or sodium hydroxide (if needed) to 5.4 units. The different timepoint samples were then diluted 6.67% w/w and their viscosity measured with a gravity pipette at 60° C. These measured viscosities at 0, 1, 2, 3, 6, 22 and 23 hours were measured to be 24.95, 31.11, 28.99, 27.15, 23.04, 12.53, and 11.44 mP respectively.

The second fraction of the initial gelatin aqueous solution was used a control example and was processed alongside of the inventive example. This fraction had its pH of adjusted to 5.4 using sodium hydroxide. The solution (covered) was then placed into the same 85° C. water bath as used with the inventive method fraction. Throughout the thermal hold period, samples of the control gelatin solution were extracted for analysis at various timepoints. Once removed from the 85° C. solution, the timepoint samples were cooled to between 55-60° C. To compare the inventive method and control example, the same dried collagen peptide composition used in the inventive method was added produce a solution with 11% concentration of reactive peptides, with the amount required being calculated from the measured mass of the timepoint sample and its gelatin concentration. Once added the dried collagen peptides were allowed to fully dissolve. The solutions were then diluted to approximately 10% w/w, the pH was readjusted with sulfuric acid or sodium hydroxide (if needed) to 5.4 units. The different timepoint samples were then diluted to 6.67% w/w and their viscosity measured with a gravity pipette at 60° C. These measured viscosities at 0, 1, 2, 3, 6, 22 and 23 hours were measured to be 34.95, 32.00, 31.41, 30.20, 25.59, 17.08, and 15.01 mP respectively.

Using the empirical relationship described in Example 3, equation 1 the time required to reach a target viscosity of 25 mP is calculated to be 4.3 hours using the inventive method and 7.4 hours using the control example. The time required to reach a target viscosity of 20 mP is calculated to be 8.4 hours using the inventive method and 14.7 hours using the control example. The time required to reach a target viscosity of 15 mP is calculated to be 15.9 hours using the inventive method and 27.6 hours using the control example.

Samples of each solution were then diluted further to about 0.1%. These samples were saved in a refrigerator at 4-8° C. temporarily until all the timepoints for a particular pH degradation run were collected. The retained samples were then analyzed using size exclusion chromatography. The size exclusion chromatography method used was the same as described in Example 3. TABLE M provides the molecular weight distributions produced by the inventive method at a pH of 5.4 and demonstrates an increased rate of microgel degradation compared the control example.

Review of the size exclusion chromatographs finds that the inventive process reduced the microgel and HMW fraction of the gelatin at a significantly higher rate than from thermal treatment alone. Using a process pH of 5.4 and 3.0 hours of reaction time, reduced the microgel content from 2.5% to 0.6% using the present invention, while a thermal process only reduced the microgel content from 2.4% to only 1.6%. After 6.0 hours of reaction time, the microgel content was 0.3% using the present invention, while a thermal process only reduced the microgel content to 1.1%.

TABLE M

| Process | Reaction | | | | | |
|---|---|---|---|---|---|---|
| (pH held | Time | | | α | sub-α | |
| at 5.4) | (hrs) | Microgel | HMW | peak | peak | LMW |
| Present | 0.0 | 2.5% | 35.8% | 14.6% | 34.8% | 12.3% |
| Invention | 1.0 | 1.5% | 32.8% | 14.9% | 38.4% | 12.4% |
| | 2.0 | 1.1% | 30.2% | 14.8% | 41.4% | 12.5% |
| | 3.0 | 0.8% | 27.8% | 14.7% | 44.1% | 12.7% |
| | 6.0 | 0.3% | 21.4% | 13.9% | 51.4% | 13.1% |
| | 22.0 | 0.0% | 5.6% | 8.1% | 69.9% | 16.4% |
| | 27.0 | 0.0% | 3.8% | 6.7% | 72.0% | 17.5% |
| Control | 0.0 | 2.4% | 35.7% | 14.6% | 35.1% | 12.3% |
| | 1.0 | 2.0% | 33.4% | 14.3% | 37.1% | 13.3% |
| | 2.0 | 1.8% | 32.7% | 14.4% | 39.0% | 12.2% |
| | 3.0 | 1.6% | 31.2% | 14.1% | 40.5% | 12.5% |
| | 6.0 | 1.1% | 27.5% | 13.4% | 44.8% | 13.2% |
| | 22.0 | 0.1% | 14.5% | 10.9% | 59.0% | 15.6% |
| | 27.0 | 0.0% | 12.1% | 10.2% | 61.6% | 16.0% |

### Example 5

This example demonstrates the ability of the reactive peptides to increase the rate of viscosity and microgel degradation of a Type A gelatin using glycine as the reactive peptides.

A dried acid processed pig skin gelatin (Bloom of 275 g and a 6.67%/60° C. viscosity of 41.8 mP) was dissolved into 55-60° C. water to produce an 20% w/w aqueous gelatin solution. This solution was then divided into 2 fractions. The first fraction of this initial gelatin solution was used to demonstrate the inventive method. This fraction was combined with 11% reactive peptides which were comprised of a dried glycine. Once the dried collagen peptides were fully dissolved, the pH of the solution was adjusted to 5.4 using sodium hydroxide. The solution (covered) was then placed into an 85° C. water bath. Throughout the thermal hold period, samples of the gelatin solution were extracted for analysis at various timepoints. Once removed from the 85° C. solution, the timepoint samples were cooled to between 55-60° C. The solutions were then diluted to approximately 10% w/w, the pH was readjusted with sulfuric acid or sodium hydroxide (if needed) to 5.4 units. The different timepoint samples were then diluted to 6.67% w/w and their viscosity measured with a gravity pipette at 60° C. These measured viscosities at 0, 1, 2, 3, 5, 23 and 28 hours were measured to be 35.53, 32.89, 30.20, 27.46, 24.00, 12.17 and 9.59 mP respectively.

The second fraction of the initial gelatin aqueous solution was used a control example and was processed alongside of the inventive example. This fraction had its pH of adjusted to 5.4 using sodium hydroxide. The solution (covered) was then placed into the same 85° C. water bath as used with the inventive method fraction. Throughout the thermal hold period, samples of the control gelatin solution were extracted for analysis at various timepoints. Once removed from the 85° C. solution, the timepoint samples were cooled to between 55-60° C. To compare the inventive method and control example, the same dried glycine used in the inventive method was added produce a solution with 11% concentration of reactive peptides, with the amount required being calculated from the measured mass of the timepoint sample and its gelatin concentration. Once added the dried glycine was allowed to fully dissolve. The solutions were then diluted to approximately 10% w/w, the pH was readjusted with sulfuric acid or sodium hydroxide (if needed) to 5.4 units. The different timepoint samples were then diluted to 6.67% w/w and their viscosity measured with a gravity pipette at 60° C. These measured viscosities at 0, 1, 2, 3, 5, 23 and 28 hours were measured to be 36.40, 34.07, 33.48, 31.11, 16.48 and 13.95 mP respectively.

Using the empirical relationship described in Example 3, equation 1 the time required to reach a target viscosity of 25 mP is calculated to be 4.6 hours using the inventive method and 8.8 hours using the control example. The time required to reach a target viscosity of 20 mP is calculated to be 8.5 hours using the inventive method and 15.1 hours using the control example. The time required to reach a target viscosity of 15 mP is calculated to be 15.0 hours using the inventive method and 25.8 hours using the control example.

Samples of each solution were then diluted further to about 0.1%. These samples were saved in a refrigerator at 4-8° C. temporarily until all the timepoints for a particular pH degradation run were collected. The retained samples were then analyzed using size exclusion chromatography. The size exclusion chromatography method used was the same as described in Example 3. TABLE N provides the molecular weight distributions produced by the inventive method at a pH of 5.4 and demonstrates an increased rate of microgel degradation compared the control example.

Review of the size exclusion chromatographs finds that the inventive process reduced the microgel and HMW fraction of the gelatin at a significantly higher rate than from thermal treatment alone. Using a process pH of 5.4 and 3.0 hours of reaction time, reduced the microgel content from 4.3% to 1.1% using the present invention, while a thermal process only reduced the microgel content from 4.1% to only 2.4%. After 5.0 hours of reaction time, the microgel content was 0.5% using the present invention, while a thermal process only reduced the microgel content to 1.8%.

TABLE N

| Process | Reaction | | | | | |
|---|---|---|---|---|---|---|
| (pH held | Time | | | α | sub-α | |
| at 5.4) | (hrs) | Microgel | HMW | peak | peak | LMW |
| Present | 0.0 | 4.3% | 39.8% | 15.7% | 37.9% | 2.3% |
| Invention | 1.0 | 2.5% | 37.1% | 16.2% | 41.8% | 2.4% |
| | 2.0 | 1.6% | 33.9% | 16.4% | 45.5% | 2.6% |
| | 3.0 | 1.1% | 31.5% | 16.3% | 48.3% | 2.7% |
| | 5.0 | 0.5% | 26.1% | 15.9% | 54.2% | 3.3% |
| | 23.0 | 0.0% | 4.4% | 7.6% | 79.7% | 8.3% |
| | 28.0 | 0.0% | 2.8% | 6.0% | 81.3% | 10.0% |
| Control | 0.0 | 4.1% | 39.7% | 15.8% | 38.2% | 2.2% |
| | 1.0 | 3.0% | 38.2% | 16.0% | 40.5% | 2.3% |
| | 2.0 | 2.6% | 36.1% | 15.6% | 42.4% | 3.2% |
| | 3.0 | 2.4% | 35.2% | 15.6% | 44.3% | 2.6% |
| | 5.0 | 1.8% | 32.3% | 15.2% | 47.8% | 2.9% |
| | 23.0 | 0.1% | 15.3% | 11.9% | 66.8% | 5.9% |
| | 28.0 | 0.0% | 12.7% | 11.1% | 69.4% | 6.7% |

### Example 6

This example demonstrates the partial removal the reactive peptides after they have been used to reduce the viscosity and microgel concentration of a gelatin composition. A gelatin composition derived from limed bovine bone raw material was produced following the procedure described in Example 3. The viscosity of the gelatin composition was 17.8 mP as measured in at 6.67% and 60° C. This gelatin composition was dissolved into water to form a 5% w/w aqueous gelatin solution and was held at a temperature of 58° C. The solution was recirculated through a crossflow ultrafiltration chamber that utilized a 5,000 Da membrane cutoff (Vivaflow® 200 manufactured by Sartorius). The permeate solution was collected. The recirculation continued for approximately 35 minutes. The concentration of the aqueous gelatin solution was increased to approximately 15.4% w/w. Samples of the original 5% w/w aqueous gelatin solution, the concentrated gelatin solution and the collected permeate solution were analyzed by size exclusion chromatography. The microgel, HMW region, a region, sub-a region and LMW regions of the starting gelatin composition was 0.2%, 12.1%, 12.8%, 60.4%, and 14.5% respectively. The microgel, HMW region, a region, sub-a region and LMW regions of concentrated gelatin solution was 0.2%, 12.9%, 13.7%, 64.2%, and 9.0% respectively. The microgel, HMW region, a region, sub-a region and LMW regions of gelatin composition, ultrafiltration permeate was 0.0%, 0.0%, 0.0%, 0.1%, and 99.9% respectively. The amount of LMW present in the concentrated gelatin solution was decreased by 38%, while the chromatograph describing the higher molecular weight regions remained consistent with that of the original gelatin solution. The permeate solution was comprised of the reactive peptides utilized during the preparation of the gelatin composition. This example demonstrates that the amounts of reactive peptides present in a gelatin composition can be reduced. The reactive peptides in the permeate could be further concentrated. The collected permeate containing the reactive peptides could also be reused to extract gelatin from collagen containing raw materials.

### Example 7

This example demonstrates the ability of the different amounts of reactive peptides to increase the rate of viscosity and microgel degradation of a Type A gelatin.

A dried acid pretreated bovine bone gelatin (Bloom of 288 g and a 6.67%/60° C. viscosity of 40.4 mP) was dissolved into 55-60° C. water to produce an 20% w/w aqueous gelatin solution. This solution was then divided into 2 fractions. The first fraction of this initial gelatin solution was used to demonstrate the inventive method. This fraction was combined to produce a setpoint concentration of reactive peptides which were comprised of a dried collagen peptide composition, produced from a limed bovine bone gelatin, and had an average molecular weight of 650 Da. Once the dried collagen peptides were fully dissolved, the pH of the solution was adjusted to 5.0 using sulfuric acid. The solution (covered) was then placed into an 85° C. water bath. Throughout the thermal hold period, samples of the gelatin solution were extracted for analysis at various timepoints. Once removed from the 85° C. solution, the timepoint samples were cooled to between 55-60° C. The different timepoint samples were then diluted to roughly a 10% w/w concentration. The pH of each specific timepoint solution was adjusted to 5.4 using sodium hydroxide. The timepoint samples were then further diluted to 6.67% w/w and their viscosity measured with a gravity pipette at 60° C. These measured viscosities for the inventive method are provided in Table O and are reported in units of mP.

The second fraction of the initial gelatin aqueous solution was used a control example and was processed alongside of the inventive example. This fraction also had its pH adjusted to 5.0 using sulfuric acid. The solution (covered) was then placed into the same 85° C. water bath as used with the inventive method fraction. Throughout the thermal hold period, samples of the control gelatin solution were extracted for analysis at various timepoints. Once removed from the 85° C. solution, the timepoint samples were cooled to between 55-60° C. To compare the inventive method and control example, the same dried collagen peptide composition used in the inventive method was added produce a solution with the same corresponding setpoint concentration of reactive peptides as utilized during the inventive method, with the amount required being calculated from the measured mass of the timepoint sample and its concentration. Once added the dried collagen peptides were allowed to fully dissolve. The different timepoint samples were then diluted to roughly a 10% w/w concentration. The pH of the each specific timepoint solution was then adjusted to 5.4 using sodium hydroxide. The samples were then further diluted to 6.67% w/w and their viscosity measured with a gravity pipette at 60° C. These measured viscosities for the control example are provided in Table P and are reported in units of mP.

The reactive peptide concentration setpoints used were 3%, 5%, 7%, 9%, and 11%.

TABLE O

| Time | concentration setpoints | | | | |
|---|---|---|---|---|---|
| (hrs) | 3% | 5% | 7% | 9% | 11% |
| 0.0 | 36.97 | 36.40 | 34.66 | 34.95 | 34.07 |
| 1.0 | 34.66 | 32.60 | 31.11 | 30.20 | 29.60 |
| 2.0 | | 29.90 | 28.07 | 28.07 | 26.52 |
| 3.0 | 29.60 | | | 26.21 | 24.96 |
| 4.0 | | 25.90 | 24.32 | | |
| 5.0 | 25.90 | | | | |
| 6.0 | | 23.37 | 21.75 | 19.78 | 18.44 |
| 22.0 | 15.01 | | | | 11.44 |
| 22.5 | | 13.24 | | 12.53 | |
| 23.0 | | | 12.17 | | |
| 25.0 | | 12.53 | | | |
| 28.0 | 12.53 | | | 11.44 | |
| 28.5 | | | | | 10.71 |
| 29.0 | | | 11.07 | | |

TABLE P

| Time | concentration setpoints | | | | |
|---|---|---|---|---|---|
| (hrs) | 3% | 5% | 7% | 9% | 11% |
| 0.0 | 36.40 | 36.11 | 34.66 | 34.66 | 33.48 |
| 1.0 | 35.82 | 33.48 | 33.48 | 31.11 | 31.11 |
| 2.0 | | 32.00 | 30.20 | 30.20 | 27.46 |
| 3.0 | 30.51 | | | 28.68 | 26.52 |
| 4.0 | | 28.68 | 27.76 | | |
| 5.0 | 27.76 | | | | |
| 6.0 | | 25.27 | 25.27 | 24.32 | 22.72 |
| 22.0 | 16.74 | | | | 15.36 |
| 22.5 | | 15.71 | | 15.01 | |
| 23.0 | | | 15.01 | | |
| 25.0 | | 15.01 | | | |
| 28.0 | 14.31 | | | 13.60 | |
| 28.5 | | | | | 13.60 |
| 29.0 | | | 13.95 | | |

Using the empirical relationship described in Example 3, equation 1 the time required to reach a target viscosity TABLE Q shows the interpolated times required to obtain 3 different targeted viscosities using both the inventive method and the control example. The viscosity of the samples utilizing the present inventive process degraded between 23% to 108% faster than the control example using only a thermal treatment.

TABLE Q

| | Target = 25 mP | | Target = 20 mP | | Target = 15 mP | |
|---|---|---|---|---|---|---|
| | Invention Method | Control Method | Invention Method | Control Method | Invention Method | Control Method |
| concentration | Time (hrs) | Time (hrs) | Time (hrs) | Time (hrs) | Time (hrs) | Time (hrs) |
| 3% | 6.6 | 8.2 | 12.0 | 14.8 | 21.2 | 26.1 |
| 5% | 4.7 | 6.5 | 9.0 | 12.6 | 17.3 | 25.0 |
| 7% | 3.6 | 5.9 | 7.3 | 11.7 | 14.6 | 23.9 |
| 9% | 3.1 | 5.5 | 6.3 | 11.2 | 13.4 | 22.5 |
| 11% | 2.5 | 4.2 | 5.1 | 9.6 | 10.6 | 22.1 |

Samples of each solution were then diluted further to about 0.1%. These samples were saved in a refrigerator at 4-8° C. temporarily until all the timepoints for a particular degradation run were collected. The retained samples were then analyzed using size exclusion chromatography. The size exclusion chromatography method used was the same as described in Example 3.

TABLE R provides the molecular weight distributions produced by the inventive method at a reactive peptide concentration of 3% and demonstrates an increased rate of microgel degradation compared the control example.

TABLE S provides the molecular weight distributions produced by the inventive method at a reactive peptide concentration of 5% and demonstrates an increased rate of microgel degradation compared the control example.

TABLE T provides the molecular weight distributions produced by the inventive method at a reactive peptide concentration of 7% and demonstrates an increased rate of microgel degradation compared the control example.

TABLE U provides the molecular weight distributions produced by the inventive method at a reactive peptide concentration of 9% and demonstrates an increased rate of microgel degradation compared the control example.

TABLE V provides the molecular weight distributions produced by the inventive method at a reactive peptide concentration of 11% and demonstrates an increased rate of microgel degradation compared the control example.

TABLE R

| 3% | Reaction | | | | | |
|---|---|---|---|---|---|---|
| reactive | Time | | | α | sub-α | |
| peptides | (hrs) | Microgel | HMW | peak | peak | LMW |
| Present | 0.0 | 8.5% | 35.4% | 12.8% | 38.7% | 4.7% |
| Invention | 1.0 | 6.6% | 34.6% | 12.8% | 41.1% | 4.9% |
| | 3.0 | 4.1% | 32.2% | 12.6% | 45.9% | 5.2% |
| | 5.0 | 2.7% | 29.5% | 12.5% | 49.7% | 5.7% |
| | 22.0 | 0.4% | 16.9% | 10.8% | 62.9% | 9.1% |
| | 28.0 | 0.1% | 9.9% | 9.0% | 69.8% | 11.3% |
| Control | 0.0 | 8.5% | 35.5% | 12.9% | 38.9% | 4.3% |
| | 1.0 | 7.1% | 34.9% | 12.8% | 40.7% | 4.5% |
| | 3.0 | 5.2% | 33.1% | 12.5% | 44.3% | 4.9% |
| | 5.0 | 3.7% | 31.1% | 12.4% | 47.2% | 5.6% |
| | 22.0 | 0.4% | 16.9% | 10.8% | 62.9% | 9.1% |
| | 28.0 | 0.2% | 13.6% | 10.0% | 65.5% | 10.7% |

TABLE S

| 5% | Reaction | | | | | |
|---|---|---|---|---|---|---|
| reactive | Time | | | α | sub-α | |
| peptides | (hrs) | Microgel | HMW | peak | peak | LMW |
| Present | 0.0 | 6.0% | 38.1% | 9.2% | 39.4% | 7.2% |
| Invention | 1.0 | 4.0% | 36.7% | 9.2% | 42.5% | 7.6% |
| | 2.0 | 3.0% | 35.1% | 9.1% | 45.0% | 7.8% |
| | 4.0 | 1.7% | 31.6% | 8.9% | 49.2% | 8.6% |
| | 6.0 | 0.9% | 28.3% | 8.7% | 52.6% | 9.5% |
| | 22.5 | 0.0% | 10.6% | 6.3% | 66.1% | 16.9% |
| | 25.0 | 0.0% | 9.3% | 6.0% | 66.9% | 17.8% |
| Control | 0.0 | 5.3% | 38.1% | 9.2% | 39.7% | 7.5% |
| | 1.0 | 4.5% | 37.1% | 9.1% | 41.7% | 7.6% |
| | 2.0 | 3.8% | 36.1% | 8.9% | 43.4% | 7.7% |
| | 4.0 | 2.7% | 33.8% | 8.7% | 46.3% | 8.5% |
| | 6.0 | 1.9% | 31.7% | 8.6% | 48.9% | 8.9% |
| | 22.5 | 0.2% | 16.7% | 7.3% | 60.6% | 15.2% |
| | 25.0 | 0.1% | 15.3% | 7.1% | 61.6% | 15.9% |

TABLE T

| 7% | Reaction | | | | | |
|---|---|---|---|---|---|---|
| reactive | Time | | | α | sub-α | |
| peptides | (hrs) | Microgel | HMW | peak | peak | LMW |
| Present | 0.0 | 5.7% | 35.6% | 14.8% | 35.4% | 8.5% |
| Invention | 1.0 | 3.7% | 34.0% | 15.0% | 38.6% | 8.7% |
| | 2.0 | 2.6% | 32.0% | 14.9% | 41.4% | 9.1% |
| | 4.0 | 1.4% | 28.2% | 14.7% | 45.8% | 9.9% |
| | 6.0 | 0.8% | 24.6% | 14.3% | 49.4% | 10.9% |
| | 23.0 | 0.0% | 7.5% | 9.9% | 64.0% | 18.7% |
| | 29.0 | 0.0% | 5.1% | 8.4% | 65.3% | 21.2% |
| Control | 0.0 | 5.1% | 35.3% | 14.7% | 35.9% | 8.9% |
| | 1.0 | 4.5% | 34.4% | 14.6% | 37.8% | 8.6% |
| | 2.0 | 3.7% | 33.2% | 14.4% | 39.3% | 9.4% |
| | 4.0 | 2.5% | 30.9% | 14.2% | 42.1% | 10.3% |
| | 6.0 | 1.8% | 28.9% | 14.0% | 44.7% | 10.5% |
| | 23.0 | 0.1% | 14.3% | 11.9% | 56.9% | 16.7% |
| | 29.0 | 0.1% | 11.3% | 11.0% | 59.1% | 18.5% |

TABLE U

| 9% | Reaction | | | | | |
|---|---|---|---|---|---|---|
| reactive | Time | | | α | sub-α | |
| peptides | (hrs) | Microgel | HMW | peak | peak | LMW |
| Present | 0.0 | 5.7% | 34.1% | 12.6% | 37.2% | 10.3% |
| Invention | 1.0 | 4.1% | 32.5% | 12.6% | 40.6% | 10.1% |
| | 2.0 | 3.0% | 30.8% | 12.5% | 43.5% | 10.2% |
| | 3.0 | 2.2% | 28.8% | 12.4% | 46.1% | 10.4% |
| | 6.0 | 0.8% | 23.1% | 11.9% | 52.9% | 11.3% |
| | 22.5 | 0.0% | 7.4% | 8.1% | 68.6% | 15.9% |
| | 28.0 | 0.0% | 5.2% | 6.9% | 70.5% | 17.3% |
| Control | 0.0 | 5.5% | 34.1% | 12.7% | 37.4% | 10.3% |
| | 1.0 | 4.9% | 33.1% | 12.4% | 39.4% | 10.2% |
| | 2.0 | 4.4% | 32.0% | 12.2% | 40.9% | 10.6% |
| | 3.0 | 3.7% | 31.1% | 12.1% | 42.8% | 10.3% |
| | 6.0 | 2.3% | 27.7% | 11.7% | 46.9% | 11.4% |
| | 22.5 | 0.2% | 14.2% | 9.9% | 60.5% | 15.2% |
| | 28.0 | 0.1% | 11.9% | 9.3% | 62.6% | 16.1% |

TABLE V

| 11% | Reaction | | | | | |
|---|---|---|---|---|---|---|
| reactive | Time | | | α | sub-α | |
| peptides | (hrs) | Microgel | HMW | peak | peak | LMW |
| Present | 0.0 | 5.2% | 33.7% | 12.4% | 36.7% | 11.9% |
| Invention | 1.0 | 3.9% | 32.0% | 12.4% | 39.9% | 11.8% |
| | 2.0 | 2.8% | 30.1% | 12.3% | 43.1% | 11.8% |
| | 3.0 | 2.0% | 28.1% | 12.2% | 45.7% | 12.1% |
| | 6.0 | 0.7% | 21.9% | 11.6% | 52.9% | 12.9% |
| | 22.0 | 0.0% | 6.5% | 7.5% | 68.4% | 17.6% |
| | 28.5 | 0.0% | 4.2% | 6.1% | 70.4% | 19.3% |
| Control | 0.0 | 5.5% | 33.6% | 12.5% | 36.6% | 11.8% |
| | 1.0 | 4.8% | 32.6% | 12.2% | 38.6% | 11.7% |
| | 2.0 | 4.2% | 31.7% | 12.0% | 40.4% | 11.7% |
| | 3.0 | 3.7% | 30.6% | 11.9% | 41.7% | 12.1% |
| | 6.0 | 2.2% | 27.4% | 11.6% | 46.2% | 12.5% |
| | 22.0 | 0.3% | 15.0% | 10.0% | 58.9% | 15.8% |
| | 28.5 | 0.1% | 12.0% | 9.3% | 61.7% | 16.9% |

Review of the size exclusion chromatographs finds that the inventive process reduced the microgel and HMW fraction of the gelatin at a significantly higher rate than from thermal treatment alone. Using 3% reactive peptides and 5.0 hours of reaction time, reduced the microgel content from 8.5% to 2.7% using the present invention, while a thermal process only reduced the microgel content from 8.5% to only 3.7%. Using a 5% reactive peptides and 6.0 hours of reaction time, reduced the microgel content from 6.0% to 0.9% using the present invention, while a thermal process only reduced the microgel content from 5.3% to only 1.9%. Using a 7% reactive peptides and 6.0 hours of reaction time, reduced the microgel content from 5.7% to 0.9% using the present invention, while a thermal process only reduced the microgel content from 5.1% to only 1.8%. Using a 9% peptide peptides and 6.0 hours of reaction time, reduced the microgel content from 5.7% to 0.8% using the present invention, while a thermal process only reduced the microgel content from 5.5% to only 2.3%. Using a 11% peptide peptides and 6.0 hours of reaction time, reduced the microgel content from 5.2% to 0.7% using the present invention, while a thermal process only reduced the microgel content from 5.5% to only 2.2%.

### Example 8

This example demonstrates the gel strength of gelatin compositions of the present invention.

A dried limed bovine bone gelatin (Bloom of 251 g and a 6.67%/60° C. viscosity of 42.6 mP) was dissolved into 55-60° C. water to produce an 20% w/w aqueous gelatin solution. The gelatin solution was then combined with 2 and 20% reactive peptides which were comprised of collagen peptides compositions produced from limed bovine bone gelatin. The solution had its pH adjusted to 5.0 with sulfuric acid and was then placed in a water bath of 85° C. (covered with a watch glass) for several hour(s). The solution was allowed to cool to 55-60° C. Each solution then had its pH readjusted to 5.4 with sodium hydroxide. The solutions were diluted to either 12.5% or 6.67% and had their viscosities measured with a gravity pipette at 60° C. The Bloom (gel strength) of the 6.67% concentrated solution was also measured. Data on the viscosities and gel strength of example gelatin compositions of the present invention are provided in TABLE W.

TABLE W

| | Hold | | | Viscosity | Viscosity | |
|---|---|---|---|---|---|---|
| Reactive | temper- | | Hold | 6.67%/ | 12.5%/ | |
| Peptides | ature | Hold | Time | 60° C. | 60° C. | Bloom |
| (%) | (° C.) | pH | (hours) | (mP) | (cP) | (g) |
| 2% | 85 | 5.0 | 5.0 | 22.6 | 7.62 | 143 |
| 2% | 85 | 5.0 | 10.0 | 17.6 | 5.55 | 99 |
| 2% | 85 | 5.0 | 14.0 | 15.0 | 4.67 | 77 |
| 2% | 85 | 5.0 | 21.0 | 12.6 | 3.76 | 51 |
| 20% | 85 | 5.0 | 1.0 | 24.9 | 8.35 | 155 |
| 20% | 85 | 5.0 | 2.0 | 21.6 | 6.74 | 135 |
| 20% | 85 | 5.0 | 3.0 | 18.7 | 5.90 | 116 |
| 20% | 85 | 5.0 | 4.0 | 17.1 | 5.30 | 103 |
| 20% | 85 | 5.0 | 6.0 | 14.5 | 4.32 | 81 |
| 2% | 75 | 4.0 | 1.0 | 26.2 | 9.10 | 207 |
| 2% | 75 | 4.0 | 1.5 | 23.5 | 7.83 | 196 |
| 2% | 75 | 4.0 | 2.0 | 20.7 | 6.68 | 178 |
| 2% | 75 | 4.0 | 2.5 | 19.1 | 5.96 | 158 |
| 2% | 75 | 4.0 | 3.5 | 16.5 | 5.13 | 135 |
| 20% | 75 | 4.0 | 0.5 | 25.9 | 8.71 | 169 |
| 20% | 75 | 4.0 | 1.0 | 22.1 | 6.99 | 159 |
| 20% | 75 | 4.0 | 1.5 | 18.8 | 5.72 | 143 |
| 20% | 75 | 4.0 | 2.0 | 17.5 | 5.22 | 135 |
| 20% | 75 | 4.0 | 2.5 | 15.8 | 4.58 | 120 |

### Example 9

This example illustrates the improved crosslinking resistance of the present invention. All gelatin compositions used in this example were made using limed bovine bone gelatin. A 12.5% gelatin solution was prepared by dissolving 10.0 g of different gelatin compositions into 70.0 g of distilled water. 8.00 grams of each gelatin solution placed into a 25 mL beaker along with a magnetic stirring bar. Each beaker was covered and placed in a water bath at 40° C. To initiate crosslinking 2 mL of a 0.5% glutaraldehyde solution was added. After 10 seconds the samples were loaded onto a rheometer equipped with a 60 mm plate-plate fixture and vapor trap. The kinetics were analyzed by linear oscillatory shear at 40° C., a deformation of y≈0.1, and a frequency of 1 Hz. The change of the gelatin solutions' storage modulus G′ and loss modulus G″ were monitored. The initial crosslinking rate was determined from the initial linear viscosity increase at the start of the reaction. The time required for a cross linked network to form, that compassed the entire sample, was determined by the time when G′ equaled G″. The results for different gelatin compositions are provided in TABLE X.

The standard composition used was representative of a soft capsule gelatin composition common in the industry. The initial crosslinking rate for this composition was high and the network formation time short.

The Gelita RXL composition represents the prior art as described by Ref G and contains about 8-9% low molecular weight collagen peptides. This gelatin composition has an improved initial crosslinking rate and network formation time when compared to aforementioned standard gelatin composition.

The present invention, which contains about 11-12% low molecular weight collagen peptides, has a significantly improved initial crosslinking rate and is 36 times slower than the Gelita RXL composition rate. The network formation time was longer than the maximum experimental time and thus exceeded 3600 seconds. This is a significant improvement over the standard gelatin composition and Gelita RXL composition network formation time of 301 seconds and 602 seconds, respectively.

TABLE X

| | | Viscosity | | Initial | Network |
|---|---|---|---|---|---|
| | Bloom | 6.67%/60° C. | | Crosslinking | Formation |
| | (g) | (mP) | pH | Rate (mP/s) | Time (s) |
| Standard | 160 | 32.1 | 5.6 | 0.86 | 301 |
| Gelita RXL | 167 | 32.4 | 5.7 | 0.51 | 602 |
| Present | 102 | 17.7 | 5.7 | 0.014 | >3600 |
| Invention | | | | | |

### Example 10

This example demonstrates that soft capsules can be made using the using present invention and that the present invention provides a gelatin composition with superior soft capsule dissolution performance in the presence of an ingredient, known to chemically crosslink gelatin. A set of four gel masses were prepared using different gelatin compositions. The details of each gelatin composition and gel mass formulation used are provided in TABLE Y. All gelatins examined were from a limed bovine bone raw material. The gel mass formulation was selected to obtain similar gel mass viscosities and a gelatin to plasticizer ratio of 2.

TABLE Y

| | | Viscosity | | | | |
|---|---|---|---|---|---|---|
| Gelatin | Bloom | 6.67%/60° C. | | | | Gel mass formulation |
| Composition | (g) | (mP) | pH | Microgel | LMW | (gelatin:glycerol:polysorb:water) |
| Standard | 159 | 30.5 | 5.7 | 5.08% | <1.5% | 44:11:11:34 |
| Gelita RXL | 166 | 32.9 | 5.7 | 4.90% | ~8% | 44:11:11:34 |
| Lower Viscosity Blend | 174 | 25.6 | 5.4 | 2.09% | <1.5% | 46:11.5:11.5:31 |
| Present Invention | 102 | 17.8 | 5.6 | 0.33% | ~11% | 52:13:13:22 |

All capsules used the same fill components. The fill components were PEG-400 [97%], glycerol [3%], brilliant blue dye [0.5%]. Soft capsules were made using a 7.5 oval die roll running at 2.5 rpm. The capsules were tumble dried for 30 minutes, then dried at about 22° C. and 40-50% r.h. for 1 week.

The capsules were stored in a mixture of medium chain triglycerides (97.5%) and peppermint oil (2.5) in closed bottles at 40° C. Peppermint oil is a known crosslinker of gelatin capsules.

After 8 weeks the capsules were removed from the storage solution. The dissolution performance of the soft capsules was then measured by submersion in a simulated gastric fluid (USP paddle no 2, 50 rpm). Shell dissolution and fill release were monitored simultaneously at 225 nm (for gelatin shell detection) and 635 nm brilliant blue dye (fill release). The experiments were repeated for six capsules from each test condition. The average degree of capsule shell dissolution as a function of time is provided in FIG. 4. The average amount of fill release from the soft capsules as a function of time is provided in FIG. 5.

This experiment found the standard gelatin composition had the poorest dissolution performance with a significant delay in fill release, demonstrating the concentration of peppermint oil used in this example was sufficient to strongly crosslink the gelatin shell. The lower viscosity blend had the next poorest dissolution profile. Gelita RXL composition performed better than the low viscosity blend and had significantly reduced fill release times, illustrating the Gelita RXL gelatin composition was more resistant to the crosslinking effects of the peppermint oil. The present invention showed little to no delay in shell dissolution or fill release and demonstrated superior soft capsule dissolution performance.

### REFERENCES

A. G. Ward and A. Courts, “The Science and Technology of Gelatin”, Food Science and Technology: A series of monographs, 1977, Academic Press, New York. ISBN: 0 12 735050 0
R. Schrieber and H. Gareis, “Gelatine Handbook: Theory and Industrial Practice”, 2007, Wiley-VCH GmbH & Co., KGA, Weinheim. ISBN: 978-3-527-31548-2
P. Davis and B. E. Tabor, “Kinetic Study of the Crosslinking of Gelatin by Formaldehyde and Glyoxal”, 1963, Journal of Polymer Science: Part A, 1, pp 799-815.
F. Moll, H. Rosenkranz and W. Himmelmann, “The Structure of Gelatin Crosslinked with Formaldehyde”, 1974, The Journal of Photographic Science, 22:6, pp 255-261.
Ian D. Robinson, “Rate of Crosslinking of Gelatin in Aqueous Solution”, 1964, Journal of Applied Polymer Science, 8, pp 1903-1918.
G. A Digenis and T. B. Gold, V. P. Shah, “Cross-linking of gelatin capsules and its relevance to their in vitro-in vivo performance”, 1994, J. Pharm Sci., 83 (7) pp 915-921.
U.S. Pat. No. 7,485,323 B2, John M. Dolphin, Tom Keenan, Jason D. Russell, Wilfried Babel, “Process for making a low molecular weight gelatine hydrolysate and gelatine hydrolysate compositions”, filed May 31, 2005.
U.S. Pat. No. 6,080,843, Robert F. Rainville, Anne G. Rowlands, Deborah J. Burrows. Peter Noble, “Gelatin and Method of Manufacture”, filed Nov. 3, 1998.

1. A method to increase the rate of viscosity degradation of a gelatin, comprising the steps of:
providing an aqueous solution of gelatin;
combining the aqueous solution of gelatin with about 2 to about 20% reactive peptides (in terms of the weight fraction of the reactive peptides relative to the total dissolved protein content of the aqueous solution), wherein the reactive peptides comprise collagen peptides with an average molecular weight of about 100 to about 5,000 Da and/or amino acids;
adjusting a pH of the aqueous solution about 3.4 to about 8.0;
heating the aqueous solution to a temperature from about 60 to about 150° C.; and
holding the aqueous solution at said temperature for a period of time sufficient to degrade the viscosity of the solution to be in a range from about 15 to about 25 mP, when measured at a total dissolved protein concentration of 6.67% w/w, at a temperature of 60° C. and at a pH of 5.3 to 5.5.

2. The method of claim 1, wherein the aqueous solution of gelatin is combined with about 9 to about 13% reactive peptides (in terms of the weight fraction of the reactive peptides relative to the total dissolved protein content of the aqueous solution).

3. The method of claim 1, wherein the pH of the aqueous solution is adjusted to from about 4.5 to about 5.5.

4. The method of claim 1, wherein the aqueous solution is heated to a temperature from about 90 to about 100° C.

5. The method of claim 1, wherein the aqueous solution is held at said temperature for a period of time sufficient to degrade the viscosity of the solution to be in a range from about 17 to about 22 mP, when measured at a total dissolved protein concentration of 6.67% w/w, at a temperature of 60° C. and at a pH of 5.3 to 5.5.

6. The method of claim 1, wherein the aqueous solution is held at said temperature for less than about 24 h, for less than about 10 h, or for less than about 5 h.

7. The method of claim 1, wherein the collagen peptides are derived from Type A gelatin, Type B gelatin, collagen, collagen containing animal tissues, collagen containing animal bones, or a combination thereof.

8. The method of claim 7, wherein the collagen peptides are enzymatically hydrolyzed collagen peptides.

9. The method of claim 1, wherein the collagen peptides have an average molecular weight of about 400 to 800 Da.

10. The method of claim 1, wherein the reactive peptides further comprise peptides that are derived from hydrolyzed soy, casein, whey, or pea protein, or a combination thereof, and wherein the peptides have an average molecular weight of about 100 to about 5,000 Da.

11. The method of claim 1, wherein the amino acids are selected from alanine, arginine, asparagine, aspartic acid, cysteine, glutamine, glutamic acid, glycine, histidine, isoleucine, lysine, hydroxylysine, methionine, phenylalanine, proline, hydroxyproline, serine, threonine, tryptophan, tyrosine, valine, or a combination thereof, and/or wherein the reactive peptides comprise diglycine.

12. The method of claim 1, wherein the gelatin is obtained from bovine bone, bovine hide, porcine bone, porcine skin, fish skin, chicken bone, jellyfish or a combination thereof.

13. The method of claim 12, wherein the gelatin is either Type A gelatin, Type B gelatin, or a combination thereof.

14. The method of claim 1, wherein the concentration of the aqueous solution of gelatin is from about 5 to about 10% w/w.

15. The method of claim 1, wherein the concentration of the aqueous solution of gelatin is from about 17 to about 25% w/w.

16. The method of claim 1, wherein the concentration of the aqueous solution of gelatin is from about 30 to about 44% w/w.

17. The method of claim 1, further comprising the step of at least partially removing the reactive peptides by ultrafiltration, diafiltration, or a combination thereof after the aqueous solution has reached a viscosity of from about 15 to about 25 mP.

18. The method of claim 1, further comprising the step of recovering the at least partially removed reactive peptides by concentration using nanofiltration, reverse osmosis, evaporation or a combination thereof.

19. A gelatin composition comprising gelatin and about 2 to about 20% reactive peptides (in terms of the weight fraction of the reactive peptides relative to the total protein content of the gelatin composition), wherein the reactive peptides comprise collagen peptides with an average molecular weight of about 100 to about 5,000 Da and/or amino acids, and wherein the gelatin composition has
a viscosity from 15 to 17 mP and a Bloom value from 55 to 171 g, or
a viscosity from 17 to 19 mP and a Bloom value from 73 to 189 g, or
a viscosity from 19 to 21 mP and a Bloom value from 91 to 205 g, or
a viscosity from 21 to 23 mP and a Bloom value from 109 to 220 g, or
a viscosity from 23 to 25 mP and a Bloom value from 124 to 233 g,
when measured at a total dissolved protein concentration of 6.67% w/w, at a temperature of 60° C. and at a pH of 5.3 to 5.5.

20. The gelatin composition of claim 19, wherein the gelatin composition comprises a microgel fraction about 0.8% or less of the total protein content of the gelatin composition.

21. The gelatin composition of claim 19, wherein the gelatin composition comprises about 9 to about 13% reactive peptides (in terms of the weight fraction of the reactive peptides relative to the total protein content of the gelatin composition).

22. The gelatin composition of claim 19, wherein the pH of the composition is from 5.3 to 6.0.

23. A gelatin composition comprising gelatin and about 2 to about 20% reactive peptides (in terms of the weight fraction of the reactive peptides relative to the total protein content of the gelatin composition), wherein the reactive peptides comprise collagen peptides with an average molecular weight of about 100 to about 5,000 Da and/or amino acids, and wherein the gelatin composition has
a viscosity from 15 to 17 mP and a Bloom value from 55 to 171 g, or
a viscosity from 17 to 19 mP and a Bloom value from 73 to 189 g, or
a viscosity from 19 to 21 mP and a Bloom value from 91 to 205 g, or
a viscosity from 21 to 23 mP and a Bloom value from 109 to 220 g, or
a viscosity from 23 to 25 mP and a Bloom value from 124 to 233 g,
when measured at a total dissolved protein concentration of 6.67% w/w, at a temperature of 60° C. and at a pH of 5.3 to 5.5, and
wherein the gelatin is obtained by the method of claim 1.
